(19) United States
(12) Patent Application Publication
(10) Pub. No.: US 2010/0227895 A1
(43) Pub. Date: Sep. 9, 2010

(54) METHOD FOR TREATING EQUINE LAMINITIS

(75) Inventors: Raymond F. LeRoy, Phoenix, AZ (US); Susan E. K. Cook, Pilot Point, TX (US)

Correspondence Address: MARVIN A. GLAZER, 2141 E. HIGHLAND AVE, SUITE 155, PHOENIX, AZ, 85016, US

(73) Assignee: MPCO- THE MAGIC POWDER COMPANY, Phoenix, AZ (US)

(21) Appl. No.: 12/398,504

(22) Filed: Mar. 5, 2009

(51) Int. Cl. A61K 31/44; A61K 31/205; A61K 31/195

(52) U.S. Cl. 514/356; 514/556; 514/563; 514/567

(57) ABSTRACT

A method for the treatment and preventative care of equine laminitis includes effective administration of the amino acid L-tyrosine, alone, or in conjunction with choline bitartrate, niacin, and/or d-calcium pantothenate to regulate and restore hormonal balance, blood pressure and normal catecholamine synthesis. Tyrosine administration fosters proper vasculation in the equine's body, and specifically promotes proper circulation in and to the hoof and laminae.

### FIELD OF THE INVENTION

The present invention relates generally to the field of veterinary science and the treatment of equine livestock, such as horses, donkeys, ponies, mules and the like. More particularly, the invention relates to treatment, and preventative care, for equine laminitis.

### BACKGROUND OF THE INVENTION

Equine laminitis is a painful and devastating disease which attacks the feet of horses and other equines. It has been reported that equine laminitis is the second biggest killer of horses. When a horse is stricken with severe laminitis, euthanasia is often the only responsible option. Laminitis is the inflammation and failure of the laminae resulting in a separation of the hoof wall from the distal phalanx.

The equine hoof consists of a hoof wall that, unlike bone, is non-living tissue constructed to resist stress in all directions and never require remodeling/regrowth. In the normal horse, the distal phalanx (coffin bone) is the final digit in the leg, and attaches to the inside of the hoof. The inside of the hoof forms a wall of folded lamellae (laminae). This folding increases the surface area of the hoof wall so as to ensure better, stronger, and more flexible connection between the coffin bone and the hoof wall.

A highly vascular corium (dermis or “quick”), underlies the hoof wall. The corium consists of a dense matrix of tough, connective tissue. The corium contains a network of arteries, veins and arterioles, providing the hoof with nourishment, and is replete with many nerve endings. A lamellar corium is positioned between the inside of the hoof wall and the coffin bone, and has dermal lamellae that interlock with epidermal lamellae of the inner hoof wall. The dense matrix of connective tissue in the corium connects the basement membrane of the dermal-epidermal junction to the distal phalanx, and thus suspends the coffin bone from the inner wall of the hoof capsule.

Basal cells of the lamellae of equine inner hoof wall primarily serve to suspend the distal phalanx within the hoof capsule. The basal cells of the coronet and the sole proliferate continuously to form the hoof wall and sole. Lamellar cells, on the other hand, only proliferate when the hoof wall is injured and requires healing.

The structure of the equine hoof and foot are such that, during locomotion, roughly 90% of the shock of impact is dissipated before the shock reaches the first, distal phalanx. The dissipation of this energy is absorbed mainly by the lamellar interface.

When the laminae fail, often within forty-eight hours from the initial onset of laminitis, the coffin bone begins to reposition by rotating within the hoof. The distal phalanx begins to push down on the hoof capsule, severing and/or crushing arteries and veins, and damaging the corium of the coronet and sole.

### Pathology of Laminitis

Laminitis is described as an inflammation of the pedal laminae that form the supportive bond between the hoof and the distal phalanx. Laminitis is a complex, multi-systemic disease that results in reduced capillary perfusion, ischemia, and necrosis of the laminae. Once the lamellar foundations have been significantly damaged, continuous physiological strain on the hoof dermo-epidermal junction (bearing the animal's weight) makes repair virtually impossible.

The first indication of laminitis is often a limp in one of the horse's front legs. By checking the pulse on the main peripheral artery supplying the affected leg with blood, a handler can detect a first symptom of the disease. A soft, smooth pulse is normal, whereas a pounding, hard pulse indicates a soft tissue injury.

Laminitis begins with a 30-40 hour developmental phase. During this phase, lamellar separation is triggered, and the appearance of foot pain is not readily observable. The developmental phase usually corresponds with a problem with one or more of the animal's organ systems, such as the: gastrointestinal, respiratory, reproductive, renal, endocrine, musculoskeletal, integumentary and immune systems. Such multi-systemic aberrations modify the internal chemistry and health of the animal, and expose the lamellar tissues to danger.

One possible cause of laminitis is grain founder, the consumption of excess amounts of grain (roughly 5-8 kg by 400-450 kg horse daily). This consumption allows the starch content of grain to pass undigested into the horse's hindgut. When horses consume grain, or any other feed which contains starch or soluble carbohydrate, portions of such feed are digested by fermentation in the caecum and colon. Starch and soluble carbohydrates provide a substrate for bacteria to rapidly ferment in the horse's digestive track. This rapid fermentation causes an increased rate of volatile fatty acid production; increased molar proportion of propionic acid relative to acetic acid and butyric acid; and accumulation of lactic acid. In turn, this may lead to increased testosterone levels, lower blood pH, lower blood bicarbonate, deficit in blood bases, failure to adequately absorb nutrients, and a raised body temperature.

An acute phase follows in which the first signs of foot pain manifest themselves; recovery of the animal is still possible at this point. The clinical signs of the acute phase include: lameness, foot pain, degeneration of lamellar attachments, penetration of the sole of the hoof by the distal phalanx, recumbency, hoof wall deformation, and sloughing of the hooves. However, once foot pain is apparent, the conditions that cause severe and permanent damage begin to progress into a chronic phase.

During the chronic phase, the distal phalanx begins to reposition, permanently severing much of the laminar corium. This downward displacement of distal phalanx can be detected with good quality radiographs. During this final chronic phase, the distal phalanx and hoof wall become permanently separated by a wedge of keratinized material, also known as a lamellar wedge. The distal phalanx will also be displaced from the proximal and middle phalanges. Secondary epidermal laminar cells become deformed and elongated. The loss of hemidesmosomes, and failure of the basal cell cytoskeleton, cause the shape and behavior of the secondary laminae (SEL) to change. The basement membrane of the SEL detaches from the basal cells, compounding the injury and leading to its irreversible damage.

The enzymes metalloproteinase-2 and metalloproteinase-9 (jointly referred to as “MMP”) also contribute to the permanency of the damage, as MMP destroy key components of the lamellar attachment. Normally, MMP break down cells in order to foster regrowth, remodeling, and spatial organization by allowing classes of epidermal cells to migrate between the lamellar basement membrane, the secondary epidermal lamellae and the primary epidermal lamellae. MMP respond to stress by promoting the remodeling of bone, joints and endometrium. However, unnecessary activation of MMP exacerbates the symptoms of laminitis by destroying healthy functioning cells, causing lesions and permanent destruction of the cellular anchoring filament.

Another factor that exacerbates the symptoms of laminitis is the blocking of lamellar energy production from glucose. Acute metabolic stress (part of colitis, metritis, and carbohydrate alimentary overload) lowers glucose metabolism in the peripheral tissues. Metabolic stress is regulated by the hormones insulin, glucagon, cortisol, and adrenaline. Adrenaline promotes glucose production from other substrates, and reduces glucose consumption in the peripheral. Hoof tissues are extremely reliant on glucose. Thus, when local concentrations of glucose are rapidly decreased, accompanying a reaction to stress, lamellar separation becomes more likely. Once the lamellae are separated and weakened, mechanical forces cause further separation and repositioning of the internal workings of the foot.

### Prior Art Treatments

Despite over hundreds of years of research, equine laminitis is still not completely understood. This serious and often fatal condition has been the subject of many, varied treatments. In almost all cases, treatments have been directed to increasing blood supply to the hoof.

There are several treatments being used to care for horses with laminitis such as vasodilators, including nitroglycerin (applied transdermally), isoxsuprine (administered orally), and anti-coagulants. All current therapies suffer from difficulty in administration, non-efficacy, non-compliance by handlers, and/or complicated procedures.

Studies have shown that laminitis may begin as a vascular disease. Therefore, some treatment methods have focused on vascular control mechanisms. Vasoconstriction in the peripheral limbs has been cited as a possible first indicator of laminitis. For this reason, certain catecholamine inhibitors and antagonists have been introduced and tested for treatment of laminitis.

### Equine Metabolism

Catecholamines mediate the hormones that cause vasoconstriction. The process and manipulation of catecholamines, and catecholamine (CA) synthesis in the blood, may provide the key to controlling, preventing and treating digital vasoconstriction and laminitis in the equine. Oral and intravenous administration of catecholamine precursors have been shown to affect catecholamine synthesis and thus regulate hormone levels, thereby preventing extreme vasoconstriction or other stress mechanisms that may induce or exacerbate the onset of laminitis.

One of the major hormones affecting stress levels in the equine is adrenaline. Adrenaline is created through a multi-step process in the equine metabolism. Under normal circumstances, L-tyrosine, a precursor, is required. L-tyrosine may be a limiting factor in the production of dihydroxyphenylalanine (L-DOPA). Tyrosine (T) is converted into L-DOPA by means of the enzyme tyrosine hydroxylase (TH). In many neuronal cells, L-DOPA is decarboxylated into dopamine (DA). DA is further metabolized by beta-hydroxylase into norepinephrine (NE), and NE is methylated by phenylethanolomaine-N-methyl-transferase (PNMT) into epinephrine (E). E is also known as adrenaline.

Tyrosine hydroxylation is the rate-limiting step in the biosynthesis of DA to NE. This rate analysis is shown through the Michaelis-Menten equation. The Michaelis-Menten equation describes the relationship between the Tyrosine substrate concentration ( T) and reaction velocity. Reaction rate (v) is dependent upon the availability of the substrate T. The maximum rate (Vₘₐₓ) of enzyme mediated reaction is calculated by the Michaelis-Menten equation as follows:

$$v = \frac{{V}_{max} [ T ]}{{K}_{M} + [ T ]}$$

where K_{M} is the concentration of the Tyrosine substrate, T, at which the reaction rate reaches half of its maximum value: Vₘₐₓ/2. The apparent K_{M} of the overall reaction of T to NE is comparable to the K_{M} of T to L-DOPA. Therefore, tyrosine metabolism is the rate limiting step in the production of NE. A maximal rate of NE synthesis is achieved with concentrations of T below 10⁴ M.

L-tyrosine, T, is the specific substrate with which the enzyme TH reacts. Available levels of T regulate the rate of TH hydroxylation, and thus CA synthesis, given that TH is largely, if not fully, saturated with T in vivo.

### OBJECTS OF THE INVENTION

It is therefore an object of the present invention to provide a method of treating horses, and or other equine species, exhibiting equine laminitis.

It is another object of the present invention to provide a method for treating horses and other equines that have exhibited equine laminitis to minimize the likelihood of further outbreaks of equine laminitis.

It is yet another object of the present invention to help regulate an equine's level of digital vasoconstriction.

It is still another object of the present invention to help regulate equine hormone levels to prevent, treat and cure laminitis and the symptoms of laminitis.

It is still yet another object of the present invention to help regulate plasma catecholamine levels in equine species.

These and other objects of the present invention will become more apparent to those skilled in the art as the description of the present invention proceeds.

### SUMMARY OF THE INVENTION

Briefly described, and according to one aspect of the present invention, a method is provided for treating a horse exhibiting symptom(s) of equine laminitis. A therapeutically effective amount of tyrosine, referred to sometimes as the amino acid L-tyrosine (4-hydroxyphenylalanine) is orally administered to the horse. Preferably, therapeutically effective amounts of choline bitartrate, niacin, and/or d-calcium pantothenate are also orally administered to the horse in addition to the tyrosine.

This method provides addition of about 1.6 grams of tyrosine per day to an equine's diet. The 1.6 grams of tyrosine is preferably administered over two separate doses of approximately 0.8 grams, given twice daily. Ideally, at least 0.80 grams of tyrosine is administered each day.

In the preferred embodiment, choline bitartrate, niacin, and/or d-calcium pantothenate are also orally administered to the horse in addition to tyrosine.

The method preferably includes administration of 1.6 grams choline bitartrate daily in addition to the tyrosine; the choline bitartrate may be administered in two separate doses of 0.8 grams each, and may be administered concurrently with the tyrosine.

The method preferably includes administration of 300 milligrams niacin daily in addition to the tyrosine; the niacin may be administered in two separate doses of 150 milligrams of niacin, and may be administered concurrently with the tyrosine.

The method preferably includes administration 600 milligrams d-calcium pantothenate daily in addition to the tyrosine; and may be administered in two separate doses of 300 milligrams of d-calcium pantothenate, and may be administered concurrently with the tyrosine.

In practicing the method of the present invention, doses of each ingredient are preferably provided in accordance with the relative weight of the equine. Tyrosine may be administered at a daily dosage of approximately 3 milligrams per kilogram of the equine's weight. Choline bitartrate may be administered at a daily dosage of approximately 3 milligrams per kilogram of the equine's weight. Niacin may be administered at a daily dosage of approximately 600 micrograms per kilogram of the equine's weight. Nutrient d-calcium pantothenate may be administered at a daily dosage of approximately 1.2 milligrams per kilogram of the equine's weight.

The present invention also provides a method of providing therapy for an equine susceptible to at least one symptom of equine laminitis, the method including the oral administration of tyrosine. This method includes the administration of about 1.6 grams of tyrosine per day to an equine. The 1.6 grams of tyrosine is preferably administered over two separate doses of approximately 0.8 grams, given twice daily. In the preferred embodiment, choline bitartrate, niacin, and/or d-calcium pantothenate are also orally administered to the horse in addition to tyrosine.

The aforementioned method of providing therapy preferably includes administration of 1.6 grams of choline bitartrate daily in addition to the tyrosine; the choline bitartrate may be administered in two separate doses of 0.8 grams each, and may be administered concurrently with the tyrosine. The method also preferably includes administration of 300 milligrams of niacin daily in addition to the tyrosine; the niacin may be administered in two separate doses of 150 milligrams each, and may be administered concurrently with the tyrosine. Preferably, the method also includes administration of 600 milligrams of d-calcium pantothenate daily in addition to the tyrosine; the d-calcium pantothenate may be administered in two separate doses of 300 milligrams each, and may be administered concurrently with the tyrosine.

The method may also provides doses of each ingredient in accordance with the relative weight of the equine. Tyrosine may be administered at a daily dosage of approximately 3 milligrams per kilogram of the equine's weight. Choline bitartrate may be administered at a daily dosage of approximately 3 milligrams per kilogram of the equine's weight. Niacin may be administered at a daily dosage of approximately 600 micrograms per kilogram of the equine's weight. Nutrient d-calcium pantothenate may be administered at a daily dosage of approximately 1.2 milligrams per kilogram of the equine's weight.

### DETAILED DESCRIPTION OF THE PREFERRED EMBODIMENTS

A horse, or any equine, experiencing a symptom of laminitis will often have reduced blood flow in the affected leg(s). The veins and vascular system supplying the affected leg are constricted, preventing proper blood flow and nutrient enrichment (glucose, oxygen, etc.) required by the cells that maintain the hoof. Once the veins are constricted, the partially isolated leg tends to swell and become infected. The only solution is to restore proper blood flow to the affected region, and the arterioles therein. Restoration of blood flow alleviates swelling, combats infection, restores nutrients, and normalizes the strength and proper function of the hoof.

Deficiency in tyrosine leads to impairment of the catecholamine and hormonal system of the horse. Tyrosine deficiency triggers ailments ranging from improper hormonal regulation, to spikes in blood pressure and vasoconstriction.

By supplying a horse with tyrosine, and preferably with other complimentary compounds, one can restore the balance to the horse's hormonal and catecholamine system. Proper catecholamine synthesis leads to enhanced blood flow which prevents, treats, and cures symptoms of laminitis.

### Importance of L-tyrosine

L-tyrosine can trigger the production of catecholamines, and other stress related hormones. In particular, tyrosine is converted (in a series of sequential steps) into L-DOPA, DA, NE, and E. Catecholamines (DA, NE, and E), particularly NE and E which are produced in the adrenal glands, act as vasoconstrictors to shut-off blood supply to various parts of an animal exposed to stress. Horses (and other equine species) demonstrating signs of lameness often exhibit over-constricted venous systems, possibly due to over-reactions to stress and inability to properly regulate stress hormones. By administering effective amounts of tyrosine, vasoconstriction levels are less erratic, over-reactions are avoided, and the venous system is moderated to avoid, forestall, or stop and reverse laminitis.

In the lame horse, tyrosine levels (and the ratio of tyrosine to the enzyme TH) are too low. The neurological and hormonal system described herein acts as a negative feedback system where the precursor L-tyrosine metabolizes into catecholamines. Supplying tyrosine to correct a metabolic deficiency helps to regulate blood pressure and vasoconstriction. Catecholamine production is dependent availability of its precursor tyrosine; when TH is less than 82% saturated with T, production of catecholamines increases. High levels of saturation, such as those above 86%, can essentially shut down catecholamine production. However, when tyrosine concentration increases beyond a predetermined level, a horse's body triggers processes to shunt the excess tyrosine to the thyroid gland, finds other uses for the tyrosine, and excretes excess tyrosine. Thus, a balanced level of tyrosine saturation is preferred to mitigate restrictive properties on the venous system. The desired ratio of saturation of TH with T is generally between 82-86%. When the ratio of T saturation is boosted to these levels, venous constriction problems are alleviated.

### Pharmacology

Neurotransmitter formation is primarily dependent upon the conditions affecting the initial step of converting tyrosine to DOPA. There are numerous CA cells in the peripheral nervous system (including the legs and feet), many of which participate in sympathetic nervous system function. Virtually all the cells in the chain of sympathetic ganglia contain NE. Also some small cells in the sympathetic ganglia contain DA. Cells in the adrenal medulla, a sympathetically innervated structure, secrete E into the blood stream. The effective amounts of catecholamines produced in the adrenal medulla, and other peripheral catecholiminergic cells, more directly impact vasodilation in the animal's digits.

In the peripheral vascular system, arterioles have a dense innervation of sympathetic nervous fibers. The peripheral vascular system is the first to be altered by dietary changes in tyrosine supply, mostly because arteriole terminals have first access to available tyrosine in the horse's blood.

Neurotransmitter precursors affect transmission at specific synapses because of their pre-synaptic site of action. Unlike drugs that act directly on postsynaptic receptors, precursors are unable to affect synaptic transmission unless the pre-synaptic neuron “allows” them to do so; the neuron allows a precursor to have an effect when it continues firing at a rate without compensating for increased transmitter release. If a neuron is responsive to fluctuations in the plasma concentrations of its precursors, then it will allow increased transmitter levels to lead to increased transmitter release (by not decreasing firing rate of buffering). Accordingly, neurotransmitter precursors, such as tyrosine, have the ability to enhance neurotransmission selectively at the synapses when such enhancement is needed.

Tyrosine and choline have little or no intrinsic activity at synapses. However, it is possible to clinically affect the synapses at which they act by administering their precursors. The noradrenergic cells at the periphery are indirectly activated by low levels of tyrosine.

In order for a precursor plasma level to affect neurotransmitter synthesis: 1) the plasma levels must be able to change, 2) the precursor must be one which the body/brain cannot produce, and 3) a low-affinity enzyme must catalyze conversion of the precursor to the transmitter. Tyrosine and choline generally meet these requirements.

Tyrosine administration increases blood pressure in hypotensive animals, and decreases blood pressure in hypertensive animals. In addition, tyrosine administration before a stressor or shock does not cause NE depletion or decreased locomotion. Tyrosine can thereby effectively regulate blood pressure.

CA (and acetylcholine) synthesis is known to be affected by plasma composition and by the availability of the precursors tyrosine and choline. CAs are synthesized from a circulating precursor T. The neurons cannot make this precursor themselves, and are therefore dependent on supplementation.

CA synthesis is enhanced by T administration when given in conjunction with other treatments such as: haloperidol, cold-stress, yohimbine, prolactin, reserpine and nigrostriatal lesions. Thus, pretreatment of neurons (activation) is thought to increase the rate of CA synthesis in certain catecholaminergic neurons (which become T-responsive). T requires some kind of activation to have effect. Activated catecholaminergic neurons have an elevated response to increases in T availability. Catecholaminergic cells are generally activated under conditions of tyrosine unavailability.

Tyrosine availability substantially affects peripheral catecholaminergic cells. In general, simple DA neurons are more active than NE neurons and DA synthesis is more rapid than NE synthesis, implying that DA neurons should be more responsive to changes in tyrosine availability, but this is not the case. Noradrenergic neurons contain the enzymes TH, aromatic amino acid decarboxylase and dopamine-beta-hydroxylase (DBH) (unlike simple DA neurons which lack DBH). Thus DA formed in noradrenergic cells is rapidly beta hydroxylated to form NE.

Plasma catecholamine levels, serve as an index of peripheral catecholamine release, are increased by tyrosine administration. Increased tyrosine availability enhances catecholamine synthesis and release only during periods of increased sympathetic activity that normally occur during the course of a day. Peripheral nervous cells, unlike central nervous system (CNS) cells, are not controlled by a multi-synaptic feedback loop—thus effects of tyrosine administration on catecholamine synthesis are pronounced at the periphery and not very effective to the CNS.

The K_{M} of TH for T (the concentration of T at which the reaction proceeds at 50% of the maximal rate) is approximately 10 to 40 micro molar (μM). TH is mostly saturated with T at 50 to 100 μM. By substituting these values (K_{M}=10, T concentration of 75 μM) into the Michaelis-Menten equation, it can be calculated that the hydroxylation of tyrosine would proceed at 88% of the maximal rate. Thus if T alone controlled the CA synthesis, even large increases in its availability could only moderately increase CA synthesis.

The half-maximal rate of NE synthesis (analogous to the K_{M} of the overall reaction) occurs when the concentration of tyrosine is about 10-40 μM. The maximal rate of NE synthesis occurs with T concentrations of about 50 to 100 μM. However, firing frequency markedly affects the responsiveness of the neuron to added tyrosine.

K_{M} for tyrosine is approximately 25 μM; thus, TH is 70-80% saturated with tyrosine, under normal circumstances and stress levels. Because TH is not fully saturated with T, catecholamine synthesis can be affected by T availability and the maximum increase in CA synthesis resulting from increased T availability will not exceed about 20-30% of the normal rate. Decreases in T availability more profoundly affect CA synthesis than increases in T; thus, deficiency of T is a major problem. Moreover, increases in tyrosine levels will benefit an animal with a T deficiency, but not significantly harm an animal with a normally functioning stress balance. In addition, the effects of tyrosine administration are generally limited to the periphery, where the effect is necessary to combat some symptoms of laminitis. Whereas, increases in T availability have little effect on CA synthesis within, or release from, the CNS.

Choline can affect cholinergic transmission with the activation of TH. Release of acetylcholine is dependent on available choline. Choline's effects on the adrenal medulla are mediated by the amount of transmitter released per firing. Therefore, by treating an animal concurrently with choline and an agent known to accelerate splanchnic firing, causes an increase in TH activity that is greater than the sum of the increases caused by the two individual treatments. Choline administration enhances TH activity and induction of TH enhances E secretion. Choline administration thereby complements the effects of tyrosine administration.

Choline, in the form of acetylcholine, independently has an effect on muscle cells. Acetylcholine acts in the small, smooth muscles of the veins to relax them and foster vasodilation, lower blood pressure, and proper circulation in the animal's extremities.

Neurotransmission, mediated by monoamines, can be affected by the availability of precursor amino acids. Furthermore, precursors, as nutrients, can be used as though they were drugs to treat a disease characterized by neurotransmitter deficit.

### Tyrosine Availability Effect on Blood Pressure

In normo-tensive (regularly stressed) animals, administration of large doses of tyrosine produces a slight drop in blood pressure. The anti-hypertensive properties action of tyrosine in hypertensive animals is mediated by an increase in the synthesis and release of NE within the brain.

However, tyrosine administration increases the arterial pressure in hypotensive animals, and significantly raises blood pressure. The action of tyrosine is partly mediated by an increased synthesis and release of catecholamines from the adrenal medulla. Tyrosine administration increases catecholamine synthesis only in activated catecholaminergic neurons.

In the hypotensive animal, sympatho-adrenal neuronal cells are activated. Therefore, tyrosine acts to increase catecholamine release, resulting in vasoconstriction and increased blood pressure. In normo-tensive animals, where no population of catecholaminergic neurons are activated, tyrosine administration produces only minor effects on blood pressure. In hypertensive animals, ventral sympatho-inhibitory neurons are activated, and therefore tyrosine acts within the CNS to decrease sympathetic outflow, producing a fall in blood pressure. Tyrosine acts within the CNS to decrease sympathetic outflow, rather than at the sympathetic nerve terminals to enhance NE release. By acting on the brain to enhance NE release and suppress sympathetic neuron activity, tyrosine decreases the sensitivity of the sympathetic neurons to changes in tyrosine availability.

### Components of Proper Administration of Substances

Niacin (niconitic acid or Vitamin B3) is also known to have effects on plasma and levels of vasodilation. Furthermore, niacin is known to affect fatty acid (metabolism) levels, plasma cholesterol levels, and thus generally regulate plasma sugar levels in the animal.

The nutrient d-Calcium Pantothenate (pantothenic acid or vitamin B5) can also act in conjunction with niacin, or alone, to effect fat metabolism, hormone production, and act as a anti-stressor for the adrenal medulla regulating adrenal cortisone levels. The nutrient d-Calcium pantothenate is required for the production of Coenzyme A, which is required for the production of energy at the cellular level. It is preferably included in practicing the present method to promote rapid conversion and metabolism of L-tyrosine, as well as to foster proper energy supply to the hoof.

The preferred form of practicing the present method will now be described for a horse weighing approximately 500 kg. In practicing the present method, the four main ingredients that are administered to prevent, treat and/or cure laminitis are: choline bitartrate, L-tyrosine, Niacin, and d-Calcium Pantothenate. These ingredients may be administered in corresponding approximate ratios of 40:41:8:16 (by weight). In the preferred embodiment, an approximately 35 day supply of the treatment includes 80 grams of choline bitartrate, 82 grams of L-tyrosine, 16 grams of niacin, and 32 grams of d-calcium pantothenate. Each dose for oral administration contains approximately 1127.5 mg of choline bitartrate, 1155.6 mg of L-tyrosine, 225.5 mg of niacin, and 451.1 mg of d-Calcium pantothenate. Preferably, this dosage is administered twice per day to an affected horse.

For the 500 kg horse, it is preferred that the horse be given at least 1.5 grams of tyrosine (3 milligrams of tyrosine per kilogram of horse's weight), 1.5 grams of choline bitartrate (3 milligrams per kilogram), 300 milligrams of niacin (600 micrograms per kilogram), and 600 milligrams of d-calcium pantothenate (1.2 milligrams per kilogram) daily.

### Monitoring Tyrosine Concentrations and Vasodilation

Methods for monitoring tyrosine concentration, and relative concentration of T with TH, in the blood are known to those skilled in the art. For instance, U.S. Pat. No. 4,284,587 to Johnson, et al. describes radioenzymatic assay of catecholamines utilizing the catechol-O-methyl transferase transfer of a methyl group from a labeled methyl donor to the catecholamine followed by isolation of the O-methylated catecholamine. Further methods, such as X-ray diffraction, H—Cl protein elimination, etc., may be used. These methods to periodically test for tyrosine levels as a preventative measure. Often this method can take days since the blood samples must be shipped out to a lab for analysis. Therefore, a more immediate method is preferably used to test for the onset of laminitis, namely, checking the arterial pulse in the major artery going into the front leg. This is preferred as a more immediate test for the need for treatment, because once the physical symptoms of laminitis manifest, it can be less than 48 hours before permanent damage or infection sets into the hoof.

The following examples further illustrate the practice of the disclosed method, but are intended in no way to limit the scope of the invention which is defined in the appended claims.

### EXAMPLE 1

A quarter-horse mare first experienced laminitis at age 5 after exposure to septic waste from a failing septic field. This first bout with laminitis lasted roughly two years. At age 9, the mare was left on a heavily grazed pasture for one day and developed laminitis, the second onset of the disease during its life, presumably from exposure to fructans in grass. Transportation of the mare exacerbated the laminitis symptoms. The mare recovered gradually, but spent a lot of time lying down. Initial dosing with tyrosine improved the condition. Tyrosine was administered daily for nearly 90 days. Hair tests revealed lowered levels of copper and iron. Subsequent administration of a tyrosine composition, with copper and iron supplementation, alleviated symptoms of laminitis within 48 hours of initial dosing, repairing dopamine/catecholamine synthesis system in the mare.

### EXAMPLE 2

A ten year-old mare had exhibited a history of laminitis. During one particular bout with laminitis, symptoms included pulsing in horse's front feet. A tyrosine composition was administered to the mare, along with feed and the analgesic phenylbutazone (commonly known as ‘bute’). Initially, the mare was fed double the recommended dosage of tyrosine (four scoops daily: two in morning and two at evening). Within hours of the first dosage, the pulsing symptoms subsided. The mare experienced a quick recovery. After this bout with laminitis, the mare was treated with the recommended dosage of tyrosine and did not re-experience laminitis.

### EXAMPLE 3

A twenty year-old gelding, a retired show hunter, experienced several bouts with laminitis. Administration of a tyrosine composition for a period of two years coincided with an absence of any further bouts of laminitis during that same time period.

While amounts of tyrosine, choline bitartrate, niacin, and d-calcium pantothenate expressed above have been expressed in terms of minimal recommended amounts per dosage (or per day), the preferred form of the method administers approximately: 1,156 milligrams of tyrosine per dose; 1,128 milligrams of choline bitartrate per dose; 226 milligrams of niacin per dose; and 451 milligrams of d-calcium pantothenate per dose. Each such dose (or “scoop”) is preferably administered twice per day.

Those skilled in the art will now appreciate that a method has been described for treating horses and other equines exhibiting equine laminitis; for providing preventative care to the equine to foster good health, and to prevent or minimize the likelihood of further outbreaks of equine laminitis; and for regulating an equine's level of digital vasoconstriction and hormone levels so as to prevent, treat and cure laminitis and the symptoms of laminitis.

The present invention has been described in conjunction with preferred embodiments thereof to facilitate the understanding of the principles and application of the invention. Such specific embodiments are not intended to limit the scope of the invention. It will be apparent to those skilled in the art that modifications may be made in the embodiments chosen for illustration without departing from the spirit and scope of the invention, as defined by the appended claims.

1. (canceled)

2. (canceled)

3. (canceled)

4. (canceled)

5. (canceled)

6. (canceled)

7. (canceled)

8. (canceled)

9. The method of claim 39 wherein the steps of administering tyrosine and administering choline bitartrate are performed concurrently with each other.

10. The method of claim 39, further comprising the step of administering to the equine at least 300 milligrams of niacin each day while the equine exhibits at least one symptom of equine laminitis.

11. (canceled)

12. (canceled)

13. (canceled)

14. The method of claim 10 wherein the steps of administering tyrosine, administering choline bitartrate, and administering niacin are performed concurrently with each other.

15. The method of claim 39, further comprising the step of administering to the equine at least 600 milligrams of d-calcium pantothenate each day while the equine exhibits at least one symptom of equine laminitis.

16. (canceled)

17. (canceled)

18. The method of claim 15 wherein the steps of administering tyrosine, administering choline bitartrate, and administering d-calcium pantothenate are performed concurrently with each other.

19. (canceled)

20. (canceled)

21. (canceled)

22. (canceled)

23. (canceled)

24. (canceled)

25. (canceled)

26. (canceled)

27. (canceled)

28. The method of claim 46 wherein the steps of administering tyrosine and administering choline bitartrate are performed concurrently with each other.

29. The method of claim 46, further comprising the step of administering to the equine at least 300 milligrams of niacin each day.

30. (canceled)

31. (canceled)

32. (canceled)

33. The method of claim 29 wherein the steps of administering tyrosine, administering choline bitartrate, and administering niacin are performed concurrently with each other.

34. The method of claim 46, further comprising the step of administering to the equine at least 600 milligrams of d-calcium pantothenate each day.

35. (canceled)

36. (canceled)

37. (canceled)

38. The method of claim 34 wherein the steps of administering tyrosine, administering choline bitartrate, and administering d-calcium pantothenate are performed concurrently with each other.

39. A method for treating an equine exhibiting at least one symptom of equine laminitis, the method comprising the steps of:
administering to the equine at least 1.6 grams of tyrosine each day while the equine exhibits at least one symptom of equine laminitis; and
administering to the equine at least 1.6 grams of choline bitartrate each day while the equine exhibits at least one symptom of equine laminitis.

40. A method for treating an equine exhibiting at least one symptom of equine laminitis, the method comprising the steps of:
administering to the equine at least 1.6 grams of tyrosine each day while the equine exhibits at least one symptom of equine laminitis; and
administering to the equine at least 300 milligrams of niacin each day while the equine exhibits at least one symptom of equine laminitis.

41. The method of claim 40 wherein the steps of administering tyrosine and administering niacin are performed concurrently with each other.

42. The method of claim 40, further comprising the step of administering to the equine at least 600 milligrams of d-calcium pantothenate each day while the equine exhibits at least one symptom of equine laminitis.

43. The method of claim 42 wherein the steps of administering tyrosine, administering niacin, and administering d-calcium pantothenate are performed concurrently with each other.

44. A method for treating an equine exhibiting at least one symptom of equine laminitis, the method comprising the steps of:
administering to the equine at least 1.6 grams of tyrosine each day while the equine exhibits at least one symptom of equine laminitis; and
administering to the equine at least 600 milligrams of d-calcium pantothenate each day while the equine exhibits at least one symptom of equine laminitis.

45. The method of claim 44 wherein the steps of administering tyrosine and administering d-calcium pantothenate are performed concurrently with each other.

46. A method of treating an equine susceptible to equine laminitis, the method comprising the step of:
administering to the equine at least 1.6 grams of tyrosine each day; and
administering to the equine at least 1.6 grams of choline bitartrate each day.

47. A method for treating an equine susceptible to equine laminitis, the method comprising the steps of:
administering to the equine at least 1.6 grams of tyrosine each day; and
administering to the equine at least 300 milligrams of niacin each day.

48. The method of claim 47 wherein the steps of administering tyrosine and administering niacin are performed concurrently with each other.

49. The method of claim 47, further comprising the step of administering to the equine at least 600 milligrams of d-calcium pantothenate each day.

50. The method of claim 49 wherein the steps of administering tyrosine, administering niacin, and administering d-calcium pantothenate are performed concurrently with each other.

51. A method for treating an equine susceptible to equine laminitis, the method comprising the steps of:
administering to the equine at least 1.6 grams of tyrosine each day; and
administering to the equine at least 600 milligrams of d-calcium pantothenate each day.

52. The method of claim 51 wherein the steps of administering tyrosine and administering d-calcium pantothenate are performed concurrently with each other.
